(19) United States
(12) Patent Application Publication
(10) Pub. No.: US 2015/0223760 A1
(43) Pub. Date: Aug. 13, 2015

(54) Screening Procedure for Identifying Risk of Arrhythmia

(71) Applicant: TO HEALTH LIMITED, London (GB)

(72) Inventor: Joshua David Greifer, London (GB)

(21) Appl. No.: 14/427,938

(22) PCT Filed: Sep. 12, 2013
(86) PCT No.: PCT/GB2013/052392

§ 371 Date: Mar. 12, 2015

(30) Foreign Application Priority Data
Sep. 12, 2012 (GB) 1216289.7

(51) Int. Cl. A61B 5/00; A61B 5/026; A61B 5/0295; A61B 5/024

(52) U.S. Cl. CPC A61B 5/7275; A61B 5/02405; A61B 5/02416; A61B 5/0261; A61B 5/0295; A61B 5/7278; A61B 5/7203; A61B 2562/0238

(57) ABSTRACT

A screening procedure and apparatus enables those at risk of cardiac arrhythmia to be identified. The procedure utilises a blood flow detector (12) in conjunction with a signal analysis unit (18) to monitor a sequence of multiple heart pulses, hence to deduce the values of a sequence of multiple heart pulse intervals, and to analyse the sequence of multiple heart pulse intervals to deduce a risk parameter whose value enables those at risk of cardiac arrhythmia to be identified. The risk parameter may be calculated from a first measure, or alternatively by combining the first measure with a second measure, where the first measure is calculated from the ratio between the variability between successive intervals in the sequence, and the mean interval value, while the second measure relates to the proportion of cases in which a long interval is followed by another long interval in the sequence.

This invention relates to a screening procedure whereby a population can be screened to identify those who are at risk of cardiac arrhythmia, and in particular atrial fibrillation. It provides both an apparatus and a method for performing this screening.

Cardiac arrhythmia, and in particular atrial fibrillation, are indications of heart problems. However, patients may have such a cardiac arrhythmia while being asymptomatic, or at any rate the symptoms may not be noticed. Even if a patient has a consultation with a doctor, the symptoms may be misinterpreted, as it is not easy to identify atrial fibrillation without use of an electrocardiogram. It would be desirable to provide a non-invasive screening procedure to identify if a person is suffering from such a cardiac arrhythmia. Where a patient is identified by the screening procedure as potentially experiencing such arrhythmia, they can be referred for more sophisticated and expensive tests such as those using an electrocardiogram.

According to the present invention there is provided a screening procedure to identify those at risk of cardiac arrhythmia, the procedure utilising a blood flow detector in conjunction with a signal analysis unit, the procedure comprising using the blood flow detector to monitor a sequence of multiple heart pulses, hence deducing the values of a sequence of multiple heart pulse intervals, and analysing the sequence of multiple heart pulse intervals by means of the signal analysis unit to deduce a risk parameter whose value enables those at risk of cardiac arrhythmia to be identified.

The blood flow detector is one that detects vascular blood flow, and may for example be a photoplethysmograph, or a pulse oximeter which incorporates a photoplethysmograph.

It will be appreciated that such a screening procedure is not satisfactory unless it provides a clear distinction between those at risk and those not at risk. Two measures have been found particularly useful in this regard. A first measure involves the ratio between the variability between successive intervals in the sequence, and the mean interval value. The variability in this case may be calculated from the standard deviation of the differences between successive intervals; and may be scaled, for example being calculated as the standard deviation divided by the square root of 2.

A second measure relates to the proportion of cases in which a long interval is followed by another long interval in the sequence, where the term “long interval” means an interval that is equal to or longer than normal, for example being equal to or longer than the modal interval. Both these measures have been found to increase, as the risk of cardiac arrhythmia is increases.

The risk parameter may for example be based on the first measure, or the risk parameter may combine both the first measure and the second measure, for example being calculated as the product of the first measure and the second measure.

The screening procedure may utilise other measures. A third measure relates to the variability of the intervals, in comparison to the mean interval value; this may be calculated as the standard deviation of the sum of the differences from the mean interval value, for successive intervals in the sequence; and this again may be scaled, for example being divided by the square root of 2. A fourth measure combines the variability between successive intervals, and the third measure, as the product of the variability between successive intervals and the third measure gives an indication of the variability in the intervals; this fourth measure tends to increase as the risk of cardiac arrhythmia increases. A fifth measure provides an indication of the extent to which successive intervals differ from the mean in terms of both the variability between successive intervals and the variability of the intervals, and again this fifth measure tends to increase as the risk of cardiac arrhythmia increases.

The sequence of heart pulse intervals that is analysed in this way is preferably a continuous sequence, for example it may be a continuous sequence during a preset period of time. Pulses may be monitored for a predetermined period of time, for example 120 seconds, or alternatively until a predetermined number of pulses have been detected. The period for which pulses are monitored may be less than 5 minutes, and may be less than 3 minutes, so the procedure is quick and does not inconvenience the people being screened.

In another aspect the present invention provides a screening apparatus to identify those at risk of cardiac arrhythmia, the apparatus comprising a blood flow detector and a signal analysis unit, such that the blood flow detector may be used to monitor a sequence of multiple heart pulses, wherein the signal analysis unit is arranged to deduce the values of a sequence of multiple heart pulse intervals, and to analyse the sequence of multiple heart pulse intervals to deduce a risk parameter whose value enables those at risk of cardiac arrhythmia to be identified.

The invention will now be further and more particularly described, by way of example only, and with reference to the accompanying drawings in which:

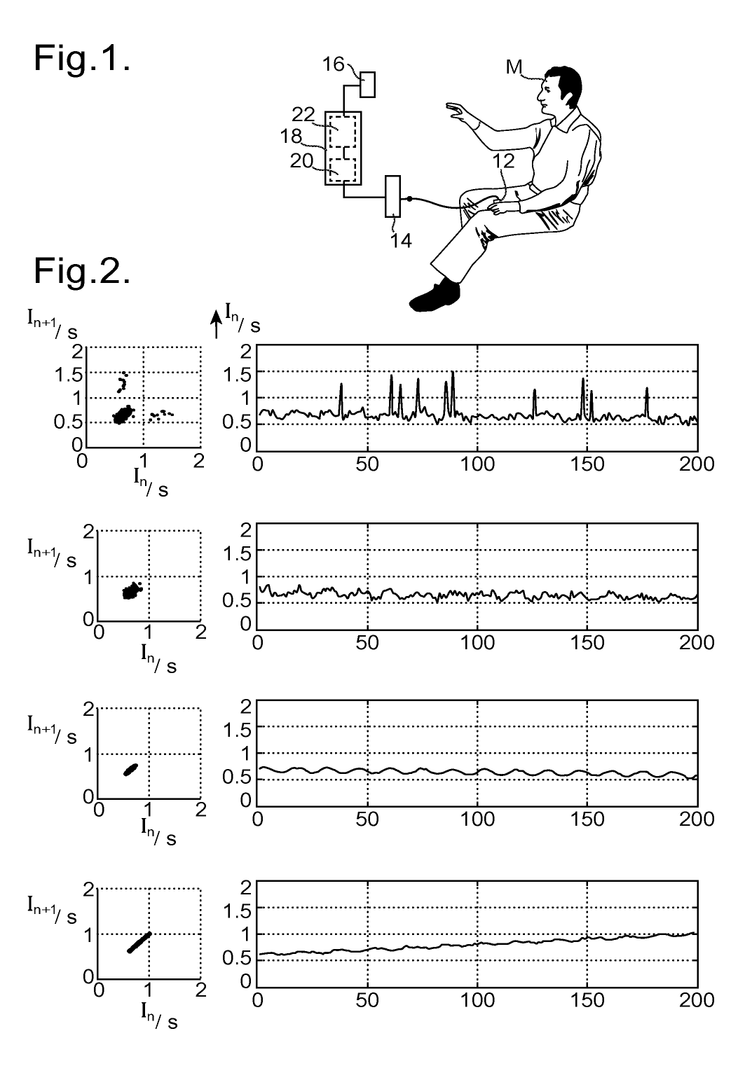

FIG. 1 shows a schematic view of apparatus of the invention;

FIG. 2 shows two different graphical representations of pulse interval variations, for four different patients;

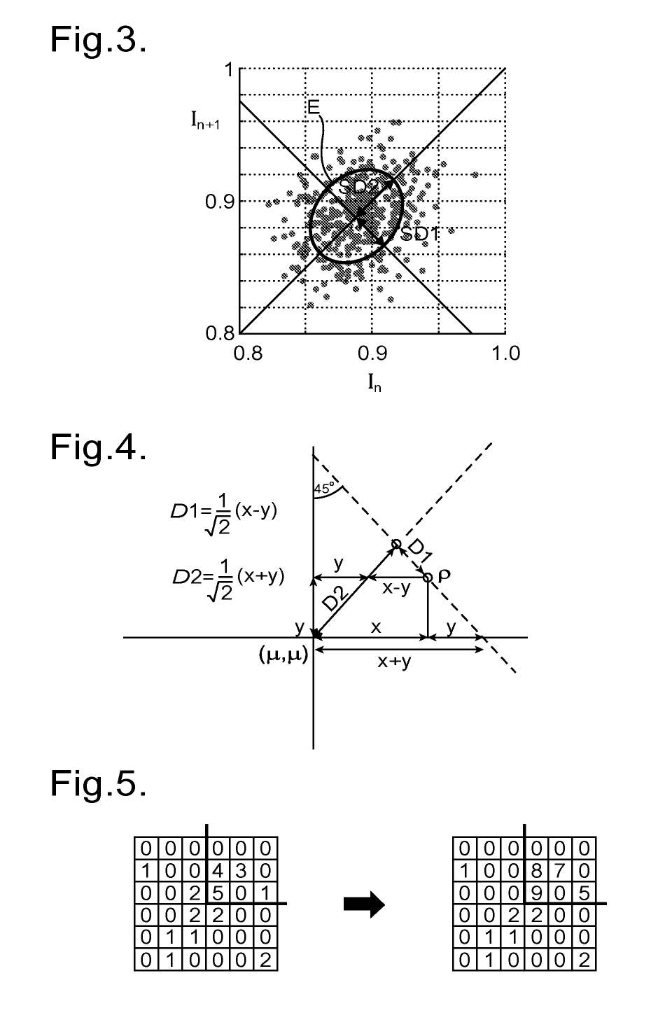

FIG. 3 shows graphically, on a Poincaré plot, measures that relate to heart rate variability;

FIG. 4 shows graphically the geometrical relationship between intervals in a Poincaré plot;

FIG. 5 illustrates graphically a calculation procedure; and

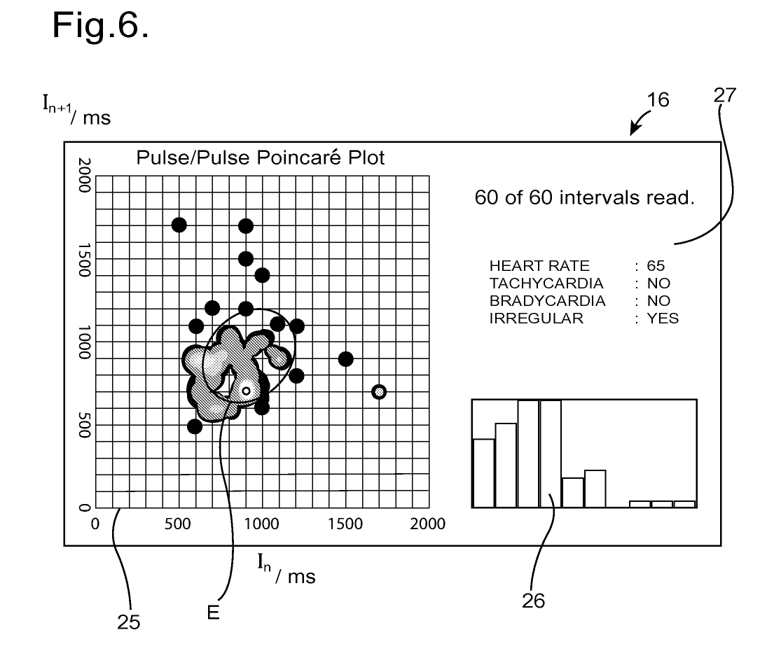

FIG. 6 shows an example display as may be produced by the apparatus of FIG. 1.

Referring now to FIG. 1 a screening apparatus 10 consists of a pulse detector 12 connected by a cable to an input module 14 acting as an interface to a signal processing unit 18. The signal processing unit 18 includes an interval detector 20 which identifies the successive peaks in the heartbeat signal provided by the pulse detector 12, and determines the durations of the successive intervals between peaks. The signal processing unit 18 also includes an irregularity detector 22 which analyses the intervals and calculates a risk parameter which enables those with cardiac arrhythmia to be distinguished from those without. The signal processing unit 18 is connected to a display 16 for displaying information about the pulse rate and pulse variability. It should be appreciated that the input module 14, the signal processing unit 18, and the display 16 may all be provided by a suitably-programmed computer, such as a laptop computer.

In use, the pulse detector 12, which may for example be a photoplethysmograph, or a pulse oximeter which incorporates a photoplethysmograph, as is arranged adjacent to a finger of a patient M, with the patient sitting down and in a relaxed state. The pulse detector 12 is arranged to detect multiple successive pulses of the patient's heart, as detected by the pulse detector 12 at the patient's finger. In a preferred arrangement measurements are taken for sufficient time to obtain data on between 60 to 150 pulse intervals, typically this taking less than two minutes. As few as 60, 80 or 100 pulse intervals may be sufficient. The input module 14 implements the protocol required to convert the data produced by the pulse detector 12 into a form which can be read by the signal processing unit 18. In one example, the resolution of the signal is seven bits, while the sample rate is 60 Hz, regulated by an internal clock.

The characteristics of the pulse detector affect the reliability of the screening procedure: an oversensitive pulse detector will incorrectly determine motion artefacts and signal noise as pulse intervals; while conversely, the detector's sensitivity should be high enough to distinguish all genuine intervals. The pulse detector may therefore be set to ignore any small maxima that follow the main pulse peak as a result of premature ventricular contractions. It has been found that a dynamic-threshold peak detector with configurable blanking period and threshold decay factor performs well. The blanking period is arranged to ensure that apparent peaks that occur a very short time period after a detected peak are not observed; and the threshold decay factor is arranged such that the threshold for detecting the next peak gradually decreases, the rate of decrease of the threshold depending on an exponentially weighted average of recent intervals. This has been found to give reliable pulse interval measurements.

Referring now to FIG. 2, this illustrates four different heart rate variation examples. In each case the data for 200 successive intervals is displayed on the right-hand side as a graph of the interval durations, I, whereas on the left-hand side the data is displayed as a Poincare plot, that is to say a two-dimensional representation of the pulse intervals, I, in which the (n+1)th interval is plotted against the preceding, nth interval, each such interval being represented by a dot. In the example shown in the top graph and plot, there are a significant number of intervals that are significantly longer than the norm, which suggests “missing” pulses due to premature ventricular contractions. In the second example there is significant arrhythmia, but no missing pulses. In the third example the intervals vary in a small amplitude sinusoidal fashion, and this is an illustration of a normal sinus rhythm in which the intervals vary in a way that is correlated with breathing. In the fourth example, in the bottom graph and plot, there is a gradual decrease in heart rate, and so a gradual increase in interval, during the observation period, and this is combined with a sinus rhythm.

The points on the Poincare plots generally fall approximately within an elliptical region centred on the mean value of the interval, μ, that is to say the centre of the elliptical region is at the point (μ, μ). Points that fall significantly outside such an ellipse may be indicative of cardiac arrhythmia, and indeed the width of the ellipse may be indicative of cardiac arrhythmia. Referring now to FIG. 3, an ellipse E may be drawn centred on the point (μ, μ), whose axes are on the two lines at 45° through that centre point. The ellipse E can be characterised by the values SD1 and SD2, which are half the lengths of the major and minor axes. The values SD1 and SD2 are defined as the standard deviations of the perpendicular distances D1 and D2 of a point to the major and minor axes.

As shown in FIG. 4 a point P is at coordinates (x, y) relative to the central point (μ, μ). Since the axes of the ellipse E are both at 45° relative to the axes representing the intervals, I, it will be appreciated that D1 is simply related to (x-y), that is to say the difference between successive intervals; and that D2 is simply related to (x+y), which is the sum of the differences from the mean interval value, for successive intervals in the sequence.

The following describes a procedure for analysing the data and determining a risk parameter. In this example the risk parameter is most sensitive to arrhythmia that arises from an atrial fibrillation.

The input data are the values of N consecutive pulse intervals, that is to say the intervals between N+1 successive pulses, taken from a patient M who is at rest. The following steps are than followed:

1) The mean value μ is determined for all the intervals.

2) The standard deviation σ is determined for all the intervals.

3) If the deviation of any intervals from μ is greater than 3×σ, those intervals are discarded (as these are likely to be artefacts)

4) A histogram is constructed from the remaining intervals, allocating the remaining intervals into Nₕ bins, where Nₕ=¼ N, the first bin accommodating the shortest interval, Imin, and the last bin accommodating the longest interval, Imax.

5) An estimated modal interval value Mo is determined for the remaining intervals. The modal interval value may be obtained by finding the bin number B of the bin with the greatest value and applying the formula:

Mo=Imin+(Imax−Imin)×(B−0.5)÷Nₕ,

where the bins are numbered from 1 to Nₕ. If there are two or more bins in which the number of intervals is equal to the greatest value, then B is taken as the lowest bin number.

6) If the value of any interval falls within ±5% of 2×Mo, that interval is discarded (as this corresponds to missing pulses due to premature ventricular contractions).

7) The standard deviation SD1 of the distances D1 is calculated for all the remaining intervals, i.e. for all the pairs of successive remaining intervals. The standard deviation SD2 of the distances D2 may also be calculated.

8) Measure 1 is calculated as the value of SD1 divided by the mean value, μ, of the intervals.

9) The interval values are quantised, in this example to 1/25 second, and any intervals longer than 2 s are excluded from further consideration. The numbers of pairs of successive intervals that fall into each quantised cell is noted. This creates a 2-dimensional matrix, distributing the data into 2500 cells (each being 1/25 s by 1/25 s).

10) The values in the cells of this matrix are multiplied by 100 and then divided by N (so as to scale the data to be independent of N).

11) The cell with the highest value is identified, and referred to as the modal cell. If more than one cell contains this value, the cell from the bottommost row is selected. If more than one cell in the bottommost row contains this value, the leftmost of these cells is selected.

12) The cells in the quadrant that has the modal cell as its bottom left hand corner are reviewed, and if any value is non-zero, 4 is added. This is illustrated in FIG. 5. This weights any tendency for long intervals to be followed by long intervals.

13) Measure 2 is then calculated as the sum of all the cell values, divided by the number of cells (2500).

14) A risk parameter is calculated by multiplying Measure 1 by Measure 2, and multiplying the result by 10000. If this is above a threshold of about 90, there is a significant risk of cardiac arrhythmia. The patient M would therefore be referred for more complex tests.

It has been found that Measure 1 typically varies between 0.010 and 0.450; while Measure 2 varies between about 0.04 and 0.18. In each case the value of the measure tends to be higher for people who have a cardiac arrhythmia. The risk parameter varies between about 4 and 700; people without cardiac arrhythmia typically have values between about 4 and 50, predominantly between 10 and 25; people with cardiac arrhythmia typically have values between 100 and 700 and predominantly between 100 and 380. Few people therefore have values near this threshold of about 90. Consequently the procedure has been found to provide a useful distinction, to enable those at risk to be identified. For example with 80 patients there was one “false negative” (with a patient whose cardiac arrhythmia was controlled by a pacemaker) and two “false positives” (where the patients had other heart problems).

The screening procedure may utilise other weighting parameters, and also may use other measures. A third measure relates to the variability of the intervals, in comparison to the mean interval value; this is the value SD2 as described above. A fourth measure is the product of SD1 and SD2, so being indicative of the area of the ellipse in the Poincare plot; this fourth measure tends to increase as the risk of cardiac arrhythmia increases.

A fifth measure provides an indication of the extent to which successive intervals differ from the mean in terms of both the variability between successive intervals and the variability of the intervals, and again this fifth measure tends to increase as the risk of cardiac arrhythmia increases. This may be calculated from the two-dimensional matrix of cell values, as derived in step 10 above. The cell values are set to 3 if they are larger than 3, and are set to 2 if they are 2 or 3. Any cell that falls predominantly in the ellipse E centred on (μ, μ) with axes SD1 and SD2 (as shown in FIG. 3) is altered: if the value is 0, set it to 1; otherwise subtract it from 3. Measure 5 is calculated as the sum of all the values of the cells, divided by the number of cells (2500). The effect of the alterations to the cell values is to lower the sum for those points that fall within the ellipse E, so that Measure 5 gives more weight to those points that fall outside the ellipse E.

Measure 5 has been found to vary between about 0.005 up to about 0.280; those with a cardiac arrhythmia tend to have values greater than about 0.050. However this does not provide such a clear distinction as does the risk parameter discussed above. It will be appreciated that the weighting described above is by way of example only, and that for determining Measure 5 an alternative weighting may be used to lower the weighting for points within the ellipse E, or to increase the weighting for those outside the ellipse E.

Referring now to FIG. 6, the display 16 may therefore be arranged to represent the data obtained from the pulse detector 12, as analysed by the signal processing unit 18. In this example the display 16 shows a Poincare plot 25 for sixty successive intervals, each axis representing pulse intervals between 0 and 2 s; the distribution of points may for example be represented by a “heat map” display, or by variations in brightness. In addition the ellipse E, generated as described above in relation to FIG. 3, is also shown. At the bottom right of the display 16 is shown a conventional histogram 26, showing the pulse intervals. At the top right hand region 27 of the display 16 are shown certain calculated parameters: in this example the heart rate is calculated as 65 per minute; there is no indication either of tachycardia or bradycardia; but the pulse rate is indicated as being irregular.

As explained above, the summary results would preferably also display the value of the calculated risk parameter, which may be calculated by multiplying Measure 1 by Measure 2, as described above, and multiplying the result by 10000. If this is above a certain threshold, there is a significant risk of cardiac arrhythmia. The value of the threshold is preferably between 75 and 125, more preferably between 80 and 105, for example about 90.

Alternatively, the risk parameter might be calculated as Measure 1 multiplied by 100. This only requires the steps 1) to 8) described above; the other steps are not required. As indicated above this typically gives a value between about 1 and 45, people without cardiac arrhythmia typically have values between 1 and 9, whereas people with cardiac arrhythmia typically have values between 11 and 45 and predominantly above 15. Few people therefore have values near a threshold value in the range 9 to 15. For example the threshold might be set at 10, 11 or 12.

It will be appreciated that a variety of different risk parameters can be calculated. It will be appreciated that the measures described above may each be scaled. For example Measure 1 is calculated above as the value of SD1 divided by the mean value, μ, of the intervals; alternatively the calculation could instead use the standard deviation of differences between successive intervals, which is larger than SD1 by a factor of the square root of 2.

What is claimed:

1. A screening procedure to identify those at risk of cardiac arrhythmia, the procedure utilising a blood flow detector in conjunction with a signal analysis unit, the procedure comprising using the blood flow detector to monitor a sequence of multiple heart pulses, hence deducing the values of a sequence of multiple heart pulse intervals, and analysing the sequence of multiple heart pulse intervals by means of the signal analysis unit to deduce a risk parameter whose value enables those at risk of cardiac arrhythmia to be identified.

2. A procedure as claimed in claim 1 wherein the blood flow detector is a photoplethysmograph, or a pulse oximeter which incorporates a photoplethysmograph.

3. A procedure as claimed in claim 1 wherein the signal analysis unit calculates a first measure from the ratio between the variability between successive intervals in the sequence, and the mean interval value.

4. A procedure as claimed in claim 3 wherein the variability between successive intervals in the sequence is calculated from the standard deviation of the differences between successive intervals.

5. A procedure as claimed in claim 3 wherein, before performing the calculations, any interval values that deviate from the mean by more than three times the standard deviation are discarded, and wherein any interval values that are within 5% of twice the modal interval are also discarded.

6. A procedure as claimed in claim 3 wherein the signal analysis unit calculates a second measure related to the proportion of cases in which a long interval is followed by another long interval in the sequence.

7. A procedure as claimed in claim 6 wherein an interval is taken as a long interval if it is equal to or longer than the modal interval.

8. A procedure as claimed in claim 1 wherein the signal analysis unit calculates the first measure as claimed in claim 3 and calculates the second measure as claimed in claim 6, and wherein the risk parameter combines the first measure with the second measure.

9. A procedure as claimed in claim 8 wherein the risk parameter is the product of the first measure and the second measure.

10. A procedure as claimed in claim 6 wherein the signal analysis unit calculates at least one of:
(a) a third measure related to the variability of the intervals, in comparison to the mean interval value;
(b) a fourth measure, which combines the variability between successive intervals, and the third measure;
(c) a fifth measure providing an indication of the extent to which successive intervals differ from the mean in terms of both the variability between successive intervals and the variability of the intervals.

11. A procedure as claimed in claim 10 wherein the third measure is calculated from the standard deviation of the sum of the differences from the mean interval value, for successive intervals in the sequence.

12. A procedure as claimed in claim 10, wherein the fifth measure is calculated by giving less weight to those pairs of successive intervals in a Poincaré plot that are within an elliptical region, E, centred on the point (mean interval, mean interval) with major and minor semi-axes oriented at 45° and of lengths equivalent to the variability between successive intervals in the sequence, and to the third measure, respectively.

13. A procedure as claimed in claim 6 wherein the sequence of heart pulse intervals that is analysed in this way is a continuous sequence.

14. A screening apparatus to identify those at risk of cardiac arrhythmia, the apparatus comprising a blood flow detector and a signal analysis unit, such that the blood flow detector may be used to monitor a sequence of multiple heart pulses, wherein the signal analysis unit is arranged to deduce the values of a sequence of multiple heart pulse intervals, and to analyse the sequence of multiple heart pulse intervals to deduce a risk parameter whose value enables those at risk of cardiac arrhythmia to be identified.

15. A screening apparatus as claimed in claim 14 wherein the signal analysis unit is adapted to perform a procedure as claimed in claim 6.
